(19) United States
(12) Patent Application Publication
(10) Pub. No.: US 2026/0360053 A1
(43) Pub. Date: Nov. 12, 2026

(54) ARTIFACT REDUCTION IN COMPUTED TOMOGRAPHY

(71) Applicant: Siemens Healthineers AG, Forchheim (DE)

(72) Inventor: Andreas SPECOVIUS, Nuernberg (DE)

(73) Assignee: Siemens Healthineers AG, Forchheim (DE)

(21) Appl. No.: 19/669,478

(22) Filed: May 6, 2026

(30) Foreign Application Priority Data
May 7, 2025 (DE) 10 2025 117 431.8

(51) Int. Cl. G06T 12/30; G06T 5/70; G06T 7/12; G06T 12/20; G06V 10/25; G06V 10/60; G16H 30/20; G16H 30/40

(52) U.S. Cl. CPC G06T 12/30; G06T 5/70; G06T 7/12; G06T 12/20; G06V 10/25; G06V 10/60; G16H 30/20; G16H 30/40

(57) ABSTRACT

A computer-implemented method for artifact reduction in CT imaging is described. In the method, CT projection measurement data is received from a region of interest, and weighted combination of the received CT projection measurement data with selectively modified raw data is performed in the raw data space. The combination-weighting factors are determined by masking pixels of an X-ray detector of a CT system used for CT imaging within the metal shadow of a metal object present in the region of interest. Artifact-reduced CT image data is reconstructed based on the combined CT projection measurement data.

### CROSS-REFERENCE TO RELATED APPLICATION(S)

The present application claims priority under 35 U.S.C. § 119 to German Patent Application No. 10 2025 117 431.8, filed May 7, 2025, the entire contents of which is incorporated herein by reference.

### FIELD

One or more example embodiments of the present invention relate to a computer-implemented method for artifact reduction. One or more example embodiments of the present invention also refer to a method for CT imaging. In addition, one or more example embodiments of the present invention relate to an image reconstruction device. Furthermore, one or more example embodiments of the present invention relate to a CT system.

### BACKGROUND

In computed tomography (CT), an object to be examined is irradiated with X-ray photons. Metals, which in clinical contexts are usually present in the form of implants or medical equipment, strongly attenuate X-ray photons in their energy range and produce a so-called metal shadow. The count rate measured at the X-ray detector within the metal shadow can therefore be significantly reduced, even for extremely short penetration depths. Depending on the size of the metal components, there may also be a large-scale absence of any X-ray photons on the X-ray detector across many X-ray detector pixels. In these detector regions, the low count rate results in “poor” photon statistics, wherein even the smallest differences in the count rate have a dramatic effect on image reconstruction. This leads to artifacts in the image data space, which manifest as straight streak artifacts traversing the entire image and passing through the metal image region.

A signal-dependent filter somewhat mitigates the effects of the unfavorable photon statistics, but does not eliminate them.

Another conventional approach consists in replacing the metal-penetrated regions in the raw data using normalized linear interpolation. Back-projected slices are used as input data or input slices for this image-based approach. To recover edge information lost through interpolation, high frequencies are extracted from the uncorrected input slices and inserted into the final image. In fact, with this so-called frequency split normalized metal artifact reduction (FS-NMAR) method, which is used to preserve fine anatomical structures, high-frequency artifacts are reinserted into the final image from the initial backprojection, so that the final image still shows high-frequency metal artifacts.

### SUMMARY

Therefore, the object is to obtain CT image data with even further reduced metal artifacts, even at high image frequencies.

This object is achieved by a computer-implemented method for artifact reduction as claimed in claim 1, a method for CT imaging as claimed in claim 6, an image reconstruction device as claimed in claim 7 and a CT system as claimed in claim 9.

In the computer-implemented method for artifact reduction in CT imaging according to one or more example embodiments of the present invention, CT projection measurement data is received from a region of interest.

Weighted combination of the received CT projection measurement data with selectively modified raw data in the raw data space is performed. The raw data space should be understood as meaning the projection measurement data space. The “weighting” locally controls the strength of the correction and ensures that the correction is substantially concentrated on the region in the raw data space that is correlated with the metal object localized in the image data space via a forward projection. Here, “substantially” is intended to indicate that there is a transition region between the subregion correlated with the localized metal object in the raw data space, also referred to as the mask region, and the remainder of the data space in which the weighting of the correction is between “1” and “0” in order to achieve a continuous smooth transition between the mask region and the surrounding area in the raw data space.

The projection measurement data comprises the measurement data captured by the X-ray detector of a CT system which comprises projections of a predetermined region of interest from different directions. Pixels of an X-ray detector of a CT system used for CT imaging are localized and masked within the metal shadow of a metal object present in the region of interest and hence masked CT projection measurement data is generated. The “pixels” comprise pixels or voxels defined in the raw data space. These pixels can be assigned to individual pixels of the X-ray detector, but they can also combine several of these X-ray detector pixels depending upon the desired resolution in the raw data space. “Pixel masking” should be understood as meaning that a region in the raw data space that is assignable to the metal object is segmented. As will be explained later, the raw data present in this mask region is replaced in a subsequent step by combined CT projection measurement data. Masking is preferably performed based on individual measurements (readings) of the rotating part of the CT system in the raw data space, preferably in a format of raw data or CT projection measurement data which in each case depicts an area in which the rows and columns are represented by detector rows and detector channels of the X-ray detector used. The aforementioned pixels in the raw data space are thus directly assigned to pixels of an X-ray detector of the CT system. The imaging comprises a plurality of the aforementioned measurements which are recorded from different directions during rotation of the rotating part of the CT system.

Furthermore, the received CT projection measurement data is selectively modified for the purpose of suppressing local fluctuations in the count rate, thereby calculating selectively modified CT projection measurement data, also referred to as smoothed CT projection measurement data. This selective modification aims to reduce fluctuation while preserving the local mean of the count rate.

In addition, appropriate combination-weighting factors are calculated with which the captured or received CT projection measurement data is weighted and combined with the smoothed CT projection measurement data, thereby generating combined CT projection measurement data. Combination is performed by weighted addition of the smoothed CT projection measurement data with the originally received CT projection measurement data based on the calculated combination-weighting factors. With this technique, pixels of the individual projections far from the metal shadow are not modified, so that the information contained therein is not modified. In addition, there is also no loss of high-frequency image information, since image-based artifact reduction by filtering the image rate is not mandatorily required. Finally, artifact-reduced CT image data is reconstructed based on the combined CT projection measurement data. The artifact-reduced CT image data is preferably reconstructed based on filtered backprojection of the combined CT projection measurement data from the raw data space into the image data space.

Advantageously, processing the raw data in the raw data space reduces high-frequency metal artifacts that are attributable to local fluctuations in the count rate in the region of the metal object mapped in the raw data space. The aforementioned correction of the CT projection measurement data in the raw data space advantageously preserves information present in the image data space that is superimposed by the metal artifacts in the image data space. This is due to the fact that the correction and combination take place in the raw data space and the raw data correlated with the information in the image data space—which superimposes the metal artifacts present in the uncorrected image—is located at a different position in the raw data space than the metal object or the raw data assigned to the metal object. Advantageously, the effect of a fluctuating count rate due to photon statistics with a locally low total number of photons is reduced or even completely compensated.

In the method, according to one or more example embodiments of the present invention, for performing CT imaging, CT projection measurement data is acquired from a region of interest and the method, according to one or more example embodiments of the present invention, for artifact reduction is applied to the acquired CT projection measurement data. Advantageously, the image quality of CT image data depicting a metal object is improved in CT imaging in the manner described above.

The reconstruction device (also referred to as a reconstruction facility) according to one or more example embodiments of the present invention has an input interface for receiving CT projection measurement data from a region of interest.

The reconstruction device according to one or more example embodiments of the present invention also comprises a masking unit. The masking unit is configured to generate localization and masking of pixels of an X-ray detector of a CT system used for CT imaging within the metal shadow of a metal object present in the region of interest and to generate masked CT projection measurement data.

Advantageously, due to the localization, the masking is limited to those regions in the raw data space that, as a result of shadowing caused by the metal object, exhibit uncertain statistics with a low total number of photons. Unmasked regions in the raw data space are fully preserved, in particular during the reconstruction of image data based on the combined CT projection measurement data.

The reconstruction device according to one or more example embodiments of the present invention also comprises an averaging unit for ascertaining smoothed CT projection measurement data based on the received CT projection measurement data.

The reconstruction device according to one or more example embodiments of the present invention also has a weighting unit for calculating appropriate combination-weighting factors based on the masked CT projection measurement data.

The combination-weighting factors comprise weighting factors w with which the received CT projection measurement data PMD is combined with the smoothed CT projection measurement data APMD, i.e., weighted according to the following schema and added to form combined CT projection measurement data MIPMD:

MIPMD=w×APMD+(1−w)×PMD. (1)

In addition, the reconstruction device comprises a combination unit for performing weighted combination of the received CT projection measurement data and the smoothed CT projection measurement data using the calculated combination-weighting factors, wherein combined CT projection measurement data is generated. The combination-weighting factors w assume the value w=1 in the mask region and the value w=0 far outside the mask region. Only in the transition region between the mask region and the region far outside the mask region are location-dependent values between 0 and 1 assumed, thereby creating a continuous transition between the mask region and the surrounding area in the raw data space.

The reconstruction device according to one or more example embodiments of the present invention furthermore comprises a reconstruction unit for reconstructing artifact-reduced CT image data based on the combined CT projection measurement data. The reconstruction unit preferably operates using the filtered backprojection method of projection measurement data commonly employed in image data reconstruction. The reconstruction device according to one or more example embodiments of the present invention shares the advantages of the method, according to one or more example embodiments of the present invention, for artifact reduction in CT imaging.

The CT system according to one or more example embodiments of the present invention has a scan unit for acquiring CT projection measurement data from a region of interest. The CT system according to one or more example embodiments of the present invention also comprises a control device (also referred to as a control facility) for actuating the scan unit and a reconstruction device according to one or more example embodiments of the present invention. The CT system according to one or more example embodiments of the present invention shares the advantages of the reconstruction device according to one or more example embodiments of the present invention.

A large proportion of the aforementioned components of the reconstruction device according to one or more example embodiments of the present invention can be implemented in whole or partially in the form of software modules in a processor of a corresponding computing system, for example, by a control device of a CT system or a computer used to control such a system. A largely software-based implementation has the advantage that even computing systems previously in use can be easily retrofitted via a software update in order to operate in the manner of one or more example embodiments of the present invention.

In this respect, the object is also achieved by a corresponding computer program product with a computer program, which can be loaded directly into a computing system, with program sections for executing the steps of the method, according to one or more example embodiments, of the present invention for artifact reduction in CT imaging, in particular the steps for localizing and masking pixels of an X-ray detector of a CT system used for CT imaging within the metal shadow of a metal object present within the region of interest, for ascertaining smoothed CT projection measurement data, for calculating the combination-weighting factors, for performing weighted combination of the received CT projection measurement data and the smoothed CT projection measurement data and for reconstructing artifact-reduced CT image data based on the combined CT projection measurement data when the program is executed in the computing system. In addition to the computer program, such a computer program product can optionally comprise additional items, such as, for example, documentation and/or additional components, including hardware components, such as, for example, hardware keys (dongles etc.) for using the software.

A computer-readable medium, for example a memory stick, a hard disk or another kind of transportable or permanently installed data carrier on which the program sections of the computer program that can be read in and executed by a computing system are stored can be used for transport to the computing system or the control device and/or for storage on or in the computing system or the control device. For this purpose, the computing system can, for example, have one or more interacting microprocessors or the like.

The dependent claims and the following description in each case contain particularly advantageous embodiments and developments of the present invention. Here, in particular the claims of one claim category can be developed analogously to the dependent claims of another claim category. In addition, it is also possible in the context of the present invention for the various features of different exemplary embodiments and claims to be combined to form new exemplary embodiments.

In a preferred variant of the computer-implemented method for artifact reduction according to one or more example embodiments of the present invention, the generation of masked CT projection measurement data comprises the steps:
performing an initial provisional filtered backprojection of the CT projection measurement data, thereby obtaining initial provisional image data,
segmenting a mask region in the initial provisional image data, which is assignable to a metal object in the region of interest, thereby generating masked image data,
applying a forward projection to the masked image data, thereby generating the masked CT projection measurement data in the raw data space.

Advantageously, localization and segmentation of the metal object in the raw data space are performed based on provisional image data in which the position and boundaries of the metal object are more easily recognizable than directly in the raw data.

Likewise, segmenting a mask region preferably comprises comparing the brightness values of the pixels of the initial provisional image data with a predetermined reference value, wherein pixels with a brightness value that exceeds the predetermined reference value are ascertained as part of the mask region to be segmented.

As an alternative to locating the metal mask in the image data space and the subsequent forward projection into the raw data space, the metal mask can also be localized directly in the raw data space, for example using an AI-based method (“AI” stands for “artificial intelligence”). This alternative variant avoids the detour of a forward projection of the mask into the raw data space. However, locating the mask region directly in the raw data space is significantly more difficult and time-consuming than locating the mask region in the image data space.

In a preferred variant of the computer-implemented method for artifact reduction according to one or more example embodiments of the present invention, the smoothed CT projection measurement data is ascertained by processing the received CT projection measurement data using a smoothing method that applies a Gaussian filter with a low bandwidth, i.e., over a few pixels, preferably one to five pixels, also preferably two or three pixels, also preferably up to ten pixels. Smoothing is preferably performed by convolving the CT projection measurement data with a Gaussian curve. Here, convolution should be understood in the mathematical sense as an operation between two functions that yields a third function. More specifically, here, integration is used to generate a mean or moving average of the intensity values of several pixels, weighted using the Gaussian curve or Gaussian function.

In a preferred variant of the computer-implemented method for artifact reduction according to one or more example embodiments of the present invention, the combination-weighting factors w are ascertained by processing the masked CT projection measurement data using a Gaussian filter, thereby performing a second smoothing, this time of the masked CT projection measurement data. Herein, the parametrization of the Gaussian function is independent of the parametrization used to calculate the smoothed CT projection measurement data.

Herein, the Gaussian filter based on the Gaussian curve or Gaussian function is preferably used to “smooth” the transition between the mask region and the remaining region in the raw data space, so that, when the combination-weighting factors are applied, instead of a step-like transition in the raw data space, a continuous transition is achieved between the smoothed CT projection measurement data and the unmodified CT projection measurement data outside the mask region in the raw data space. The Gaussian filter function is applied by convolving the masked CT projection measurement data with a Gaussian function. The spatially dependent combination-weighting factors required for the weighted addition or combination are calculated based on this convolution.

The combination-weighting factors obtained in this way ensure a gradually tapering edge of the weighting in the border region of the mask region, thereby achieving a transition to the surrounding area of the mask region with a continuous gradient of the intensity values of the CT projection measurement data.

Advantageously, on the one hand, metal artifacts arising from “poor” statistics with low photon counts are suppressed and, on the other hand, artifacts newly generated by discontinuities due to an otherwise potential step-like transition between the projection measurement data within the mask region and outside the mask region are avoided.

In a preferred embodiment of the reconstruction device according to the present invention, the masking unit comprises an initial backprojection unit for performing an initial provisional filtered backprojection of the CT projection measurement data, thereby obtaining initial provisional image data. The reconstruction device according to one or more example embodiments of the present invention also comprises a segmentation unit for segmenting a mask region in the initial provisional image data which is assignable to a metal object in the region of interest, thereby generating masked image data. The reconstruction device, according to one or more example embodiments of the present invention, furthermore comprises a forward projection unit for applying a forward projection to the masked image data, thereby generating the masked CT projection measurement data. Advantageously, in this particularly preferred variant of the reconstruction device, according to one or more example embodiments of the present invention, the mask is localized in the image data space where the region in which the metal object is arranged is easy to locate.

Advantageously, therefore, localization and segmentation of the metal object in the raw data space are performed based on provisional image data in which the position and boundaries of the metal object are more easily recognizable via forward projection from the image data space into the raw data space.

### BRIEF DESCRIPTION OF THE DRAWINGS

The present invention is explained in more detail below with reference to the accompanying figures and to exemplary embodiments. The figures show:

FIG. 1 a schematic comparative representation of a CT image without artifact correction and a CT image with artifact correction,

FIG. 2 a flowchart illustrating a method for artifact reduction in CT imaging according to a first exemplary embodiment of the present invention,

FIG. 3 a flowchart illustrating a method for performing CT imaging according to an exemplary embodiment of the present invention,

FIG. 4 a schematic representation of a reconstruction device according to an exemplary embodiment of the present invention,

FIG. 5 a schematic representation of a CT system according to an exemplary embodiment of the present invention.

### DETAILED DESCRIPTION

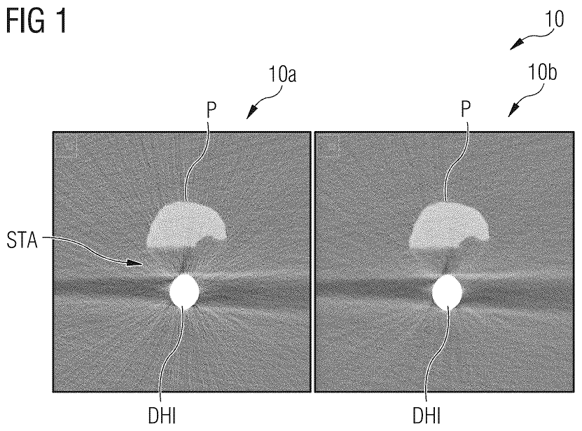

FIG. 1 is a schematic representation 10 of a first CT image 10a without artifact correction and a second CT image 10b with artifact correction. The two images 10a, 10b in each case show a phantom P and a metal object in the form of an artificial double hip implant DHI. The first image 10a shows high-frequency streak artifacts STA originating from the metal of the artificial double hip implant DHI. In the second CT-image 10b, the streak artifacts STA are suppressed by the method, according to one or more example embodiments of the present invention, for artifact reduction in CT imaging.

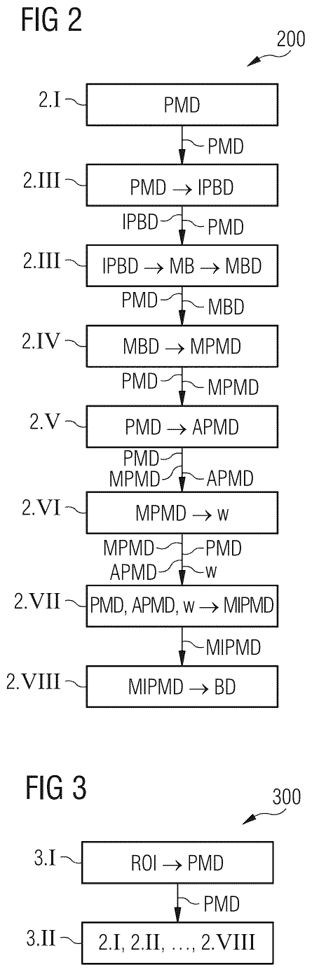

FIG. 2 shows a flowchart 200 illustrating a computer-implemented method for artifact reduction in CT imaging according to a first exemplary embodiment of the present invention.

In step 2.I, CT projection measurement data PMD is received from a region of interest ROI.

In step 2.II, an initial provisional filtered backprojection of the CT projection measurement data PMD is performed. I.e., initial provisional image data IPBD is obtained based on the CT projection measurement data PMD.

In step 2.III, a mask region MB assignable to a metal object in the region of interest ROI is segmented in the initial provisional image data IPBD. In this process, masked image data MBD is generated. Segmentation is performed by ascertaining the intensity of gray values in the initial provisional image data IPBD and by comparing the gray values with a predetermined threshold value.

Subsequently, in step 2.IV, a forward projection is applied to the masked image data MBD, thereby generating masked CT projection measurement data MPMD.

In step 2.V, selectively modified or smoothed CT projection measurement data APMD is ascertained based on the received CT projection measurement data PMD. Selectively modifying the CT projection measurement data causes local fluctuations in the count rate to be selectively attenuated while preserving the local average.

In step 2.VI, appropriate combination-weighting factors w are calculated based on the masked CT projection measurement data MPMD. The combination-weighting factors w have the value “1” in the interior of the mask region and the value “0” far outside the mask region. In a transition region between the mask region and the surrounding area in the raw data space, the combination-weighting factors w are now defined such that they enable a continuous smooth transition of the intensity values of the projection measurement data in this region.

In step 2.VII, the received CT projection measurement data PMD and the smoothed CT projection measurement data APMD ascertained in step 2.V are subjected to weighted combination using the weighting factors w ascertained in step 2.VI, thereby generating combined CT projection measurement data MIPMD.

Finally, in step 2.VIII, artifact-reduced CT image data BD is reconstructed based on the combined CT projection measurement data MIPMD. Since the source of the artifacts in the raw data space has been replaced by smoothed CT projection measurement data, the transfer of high-frequency metal artifacts into the image data space can be prevented.

FIG. 3 shows a flowchart 300 illustrating a method for performing CT imaging according to an exemplary embodiment of the present invention.

In step 3.I, CT projection measurement data PMD is acquired from a region of interest ROI.

Subsequently, the method for artifact reduction in CT imaging illustrated in FIG. 2 is applied to the acquired CT projection measurement data PMD.

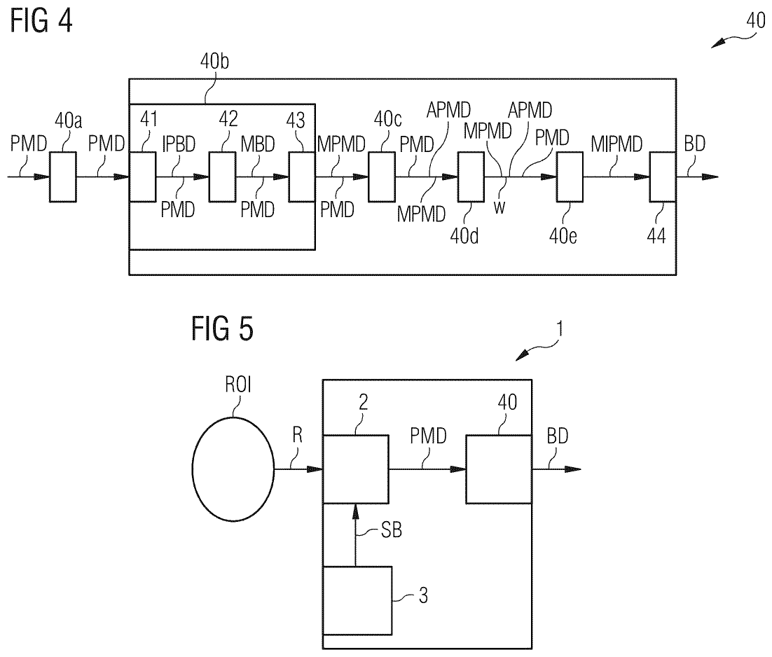

FIG. 4 is a schematic representation of a reconstruction device 40 according to an exemplary embodiment of the present invention.

The reconstruction device 40 has an input interface 40a for receiving CT projection measurement data PMD from a region of interest.

The reconstruction device 40 also comprises a masking unit 40b with which a subregion of the raw data space assignable to a metal object is masked. To this end, the masking unit 40b comprises an initial backprojection unit 41 for performing an initial provisional filtered backprojection of the CT projection measurement data PMD, thereby obtaining initial provisional image data IPBD.

In addition, the masking unit 40b has a segmentation unit 42 for segmenting a mask region in the initial provisional image data IPBD. The mask region is assignable to a metal object in the region of interest. During segmentation, masked image data MBD is generated.

The masking unit 40b also comprises a forward projection unit 43 for applying a forward projection to the masked image data MBD, thereby generating masked CT projection measurement data MPMD. The forward projection causes the mask region ascertained, localized and segmented in the image data space to be transferred to the raw data space, thereby circumventing a direct more time-consuming localization of the mask region in the raw data space.

The reconstruction device 40 also has an averaging unit 40c for ascertaining smoothed CT projection measurement data APMD based on the CT projection measurement data PMD.

In addition, the reconstruction device 40 comprises a weighting unit 40d for calculating appropriate combination-weighting factors w based on the masked CT projection measurement data MPMD.

In addition, the reconstruction device 40 comprises a combination unit 40e for performing a weighted combination of the received CT projection measurement data PMD and the smoothed CT projection measurement data APMD, thereby generating combined CT projection measurement data MIPMD.

In addition, the reconstruction device 40 comprises a reconstruction unit 44 for reconstructing artifact-reduced CT image data BD based on the combined CT projection measurement data MIPMD. As already explained, the reconstruction comprises a filtered backprojection from the raw data space into the image data space.

FIG. 5 is a schematic representation of a CT system 1 according to an exemplary embodiment of the present invention. The CT system 1 comprises a scan unit 2 for acquiring CT projection measurement data PMD from a region of interest ROI. To this end, the region of interest ROI is irradiated with X-rays R and the CT projection measurement data PMD is obtained based on sensor signals from pixels of an X-ray detector of the scan unit. In addition, the CT system 1 has a control device 3 for actuating the scan unit 2 with the aid of control commands SB. The CT system 1 also comprises a reconstruction device 40 as illustrated in FIG. 4 for reconstructing artifact-reduced CT image data BD based on the captured CT projection measurement data PMD.

Finally, it should again be noted that the methods and apparatuses described above are merely preferred exemplary embodiments of the present invention and that the present invention can be modified by a person skilled in the art without departing from the field of the present invention, to the extent that it is disclosed by the claims. It is mentioned for the sake of completeness that the use of the indefinite article “a” or “an” does not rule out the possibility of there also being more than one of the features concerned. Likewise, the term “unit” does not exclude the possibility that said unit consists of a plurality of components, which may also be spatially distributed if applicable.

Independent of the grammatical term usage, individuals with male, female or other gender identities are included within the term.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternatively, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” or “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing device or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above. Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different from that of the methods described, and/or components such as the described system, architecture, devices, circuit, and the like, may be connected or combined to be different from the above-described methods, or results may be appropriately achieved by other components or equivalents.

What is claimed is:

1. A computer-implemented method for artifact reduction in CT imaging, the computer-implemented method comprising:
receiving CT projection measurement data from a region of interest;
ascertaining masked CT projection measurement data by localizing pixels of an X-ray detector of a CT system used for the CT imaging within a metal shadow of a metal object present in the region of interest;
ascertaining smoothed CT projection measurement data based on the masked CT projection measurement data;
calculating combination-weighting factors based on the masked CT projection measurement data;
generating combined CT projection measurement data by performing a weighted combination of the CT projection measurement data and the smoothed CT projection measurement data based on the combination-weighting factors; and
reconstructing artifact-reduced CT image data based on the combined CT projection measurement data.

2. The computer-implemented method as claimed in claim 1, wherein ascertaining of the masked CT projection measurement data comprises:
obtaining initial provisional image data by performing an initial provisional filtered backprojection of the CT projection measurement data;
generating masked image data by segmenting a mask region in the initial provisional image data which is assignable to the metal object present in the region of interest; and
generating the masked CT projection measurement data by applying a forward projection to the masked image data.

3. The computer-implemented method as claimed in claim 2, wherein segmenting the mask region comprises:
comparing a brightness values of pixels of the initial provisional image data with a reference value, wherein
pixels with a brightness value that exceeds the reference value are ascertained as part of the mask region to be segmented.

4. The computer-implemented method as claimed in claim 1, wherein a mask region within the metal shadow is localized and segmented directly in the raw data space.

5. The computer-implemented method as claimed in claim 1, wherein the masked CT projection measurement data is processed by a smoothing method to generate a mask region with a border having a continuous transition.

6. A method for performing CT imaging, the method comprising:
acquiring CT projection measurement data from a region of interest; and
applying the method as claimed in claim 1 to the CT projection measurement data.

7. A reconstruction device comprising:
an input interface configured to receive CT projection measurement data from a region of interest;
a masking unit configured to ascertain masked CT projection measurement data, wherein pixels of an X-ray detector of a CT system used for CT imaging are localized within a metal shadow of a metal object present in the region of interest;
an averaging unit configured to ascertain smoothed CT projection measurement data based on the CT projection measurement data;
a weighting unit configured to calculate combination-weighting factors based on the masked CT projection measurement data;
a combination unit configured to generate combined CT projection measurement data by performing a weighted combination of the CT projection measurement data and the smoothed CT projection measurement data based on the combination-weighting factors; and
a reconstruction unit configured to reconstruct artifact-reduced CT image data based on the combined CT projection measurement data.

8. The reconstruction device as claimed in claim 7, wherein the masking unit comprises:
an initial backprojection unit configured to obtain initial provisional image data by performing an initial provisional filtered backprojection of the CT projection measurement data;
a segmentation unit configured to generate masked image data by segmenting a mask region in the initial provisional image data which is assignable to the metal object present in the region of interest; and
a forward projection unit configured to generate the masked CT projection measurement data by applying a forward projection to the masked image data.

9. A CT system having:
a scan unit configured to acquire CT projection measurement data from a region of interest;
a control device configured to actuate the scan unit; and
the reconstruction device as claimed in claim 7.

10. A computer program product comprising instructions stored on a non-transitory computer readable medium, the instructions, when executed by a computer, cause the computer to execute the computer-implemented method as claimed in claim 1.

11. A non-transitory computer-readable storage medium comprising instructions that, when executed by a computer, cause the computer to execute the computer-implemented method as claimed in claim 1.

12. The computer-implemented method as claimed in claim 4, wherein the mask region within the metal shadow is localized and segmented directly in the raw data space by an AI-based method.

13. The computer-implemented method as claimed in claim 2, wherein the masked CT projection measurement data is processed by a smoothing method to generate a mask region with a border having a continuous transition.

14. The computer-implemented method as claimed in claim 3, wherein the masked CT projection measurement data is processed by a smoothing method to generate a mask region with a border having a continuous transition.

15. The computer-implemented method as claimed in claim 4, wherein the masked CT projection measurement data is processed by a smoothing method to generate a mask region with a border having a continuous transition.

16. A CT system having:
a scan unit configured to acquire CT projection measurement data from a region of interest;
a control device configured to actuate the scan unit; and
the reconstruction device as claimed in claim 8.

17. A reconstruction device comprising:
a memory storing computer-executable instructions; and
at least one processor configured to execute the computer-executable instructions to cause the reconstruction device to
receive CT projection measurement data from a region of interest,
ascertain masked CT projection measurement data, wherein pixels of an X-ray detector of a CT system used for CT imaging are localized within a metal shadow of a metal object present in the region of interest,
ascertain smoothed CT projection measurement data based on the CT projection measurement data,
calculate combination-weighting factors based on the masked CT projection measurement data,
generate combined CT projection measurement data by performing a weighted combination of the CT projection measurement data and the smoothed CT projection measurement data based on the combination-weighting factors, and
reconstruct artifact-reduced CT image data based on the combined CT projection measurement data.

18. A non-transitory computer-readable storage medium comprising instructions that, when executed by a computer, cause the computer to execute the computer-implemented method as claimed in claim 2.

19. A non-transitory computer-readable storage medium comprising instructions that, when executed by a computer, cause the computer to execute the computer-implemented method as claimed in claim 3.

20. A non-transitory computer-readable storage medium comprising instructions that, when executed by a computer, cause the computer to execute the computer-implemented method as claimed in claim 4.
